(19) United States
(12) Patent Application Publication
(10) Pub. No.: US 2005/0084928 A1
(43) Pub. Date: Apr. 21, 2005

(54) Glutamine-auxothrophic human cells capable of producing proteins and capable of growing in a glutamine-free medium

(76) Inventors: John Robert Birch, Buckinghamshire (GB); Robert Charles Boraston, Buckinghamshire (GB); Martyn Shaw, Berkshire (GB)

Correspondence Address: NIXON & VANDERHYE, PC, 1100 N GLEBE ROAD, 8TH FLOOR, ARLINGTON, VA, 22201-4714, US

(21) Appl. No.: 10/501,777

(22) PCT Filed: Jan. 17, 2003
(86) PCT No.: PCT/EP03/00454

(30) Foreign Application Priority Data
Jan. 17, 2002 (GB) 0209777
Dec. 5, 2002 (EP) 020271896

(51) Int. Cl. C07H 21/04; C12N 5/08

(52) U.S. Cl. 435/69.1; 435/320.1; 435/366; 530/395; 536/23.5

(57) ABSTRACT

A glutamine-auxotrophic human cell transfected with an exogenous DNA sequence encoding a protein or an exogenous DNA sequence capable of altering the expression of an endogenous gene encoding a protein and an exogenous DNA sequence encoding a glutamine synthetase, wherein these exogenous DNA sequences are located on one or more than one DNA construct, said transfected cell capable of producing said protein and capable of growing in a glutamine-free medium.

The present invention relates to a novel glutamine-auxotrophic human cell capable of producing a protein and capable of growing in a glutamine-free medium. Furthermore, it relates to a novel process of producing a protein and to the use of a glutamine synthetase (GS) as a selectable marker in glutamine-auxotrophic human cells.

Protein production by mammalian cell culture is used to provide proteins for therapeutic and diagnostic applications. Today mammalian cell cultures are the preferred source of a number of important proteins for use in human and animal medicine, especially those which are relatively large, complex and glycosylated (N. B. Finter et al., in Large-Scale Mammalian Cell Culture Technology, 1990, ed. A. S. Lubiniecki, Marcel Dekker, Inc., New York).

For example, production of the human protein erythropoietin (EPO) by cell culture has been described in WO 93/09222. Human EPO has been obtained at appreciable specific production rates employing human fibroblast cells transfected with the exogenous gene coding for human EPO. In a further process for the production of human EPO (WO 94/12650), the human fibrosarcoma cell line HT1080 transfected with DNA sequences which are capable to activate the endogenous encoded EPO has been applied. A similar approach has been described in WO 99/09268. Immortalised human cells like Namalwa, Hela S3 and HT 1080 cells have been transfected with DNA sequences which are capable to activate the endogenous encoded EPO.

The cells described in WO 93/09222, WO 94/12650 and WO 99/09268 used for the production of human EPO have all been cultured in a medium containing glutamine. This is disadvantageous because when glutamine is used as an energy substrate by cultured cells, ammonia is a catabolite produced, which is cytotoxic and can inhibit cell growth. Furthermore, it can inhibit glycosylation of proteins by its effect on pH within the Golgi of the cell.

The production of high levels of tissue plasminogen activator, a glycosylated protein, has been described in WO 87/04462. GS has been used herein as amplification system for co-amplifying the gene encoding tissue plasminogen activator (tPA) in glutamine-prototrophic Chinese hamster ovary (CHO) cells by transfecting the cells with a gene encoding GS. As found in EP-A 148 605, it is disadvantageous, however, to use CHO cells for the production of a glycosylated human protein. Proteins synthesized by CHO cells may differ in their average carbohydrate composition from natural occurring glycosylated human proteins. This is due to the fact that human cells possess α2.3 sialyltransferase and α2.6 sialyltransferase enzymes. CHO cells possess only the α2.3 sialyltransferase and so cannot perform the α2.6 linkage of terminal sialic acid to the oligosaccharide moieties. CHO cells lack the enzymes for sulphation of the carbohydrate structures. CHO cells also lack an α1-3 fucosyltransferase (attaches terminal fucose residues) though do have α1-6 fucosyltransferase (attaches core fucose residues). Human cells have both fucosyltransferases. (Cumming D. A., 1991, Glycobiology Vo. 1, No. 2, 115-130, Jenkins N. and Curling E. M. A., 1994, Enzyme and Micorbial Technology Vol. 16, 354-364. Lee et al., 1989, Journal of Biological Chemistry, Vol. 264, 13848-13855). Therefore, glycosylated proteins synthezised by CHO cells may not have the desired characteristics e.g. in-vivo biological activity as the one produced in human cells.

In WO 89/10404, a method of making myeloma cells such as mouse hybridoma, mouse plasmacytoma cells and rat hybridoma cells glutamine-independent by transforming them with GS has been reported. It has been further demonstrated herein that GS can be used for co-amplification of genes encoding light and heavy chains of immunoglobulin molecules and for co-amplification of a gene encoding a fibrinolytic enzyme in a myeloma cell line. However, rodent cell lines present disadvantages, namely the attachment of N-glycolylneuraminic acid residues in place of the N-acetylneuraminic acid, the inability to carry out sulphation and the presence of the α1.3 galactosyltransferase enzyme. Oligosaccharide structures of glycoproteins synthesized in rodent cells might therefore be expected to be immunogenic in humans.

The object of the present invention is to provide an improved process which does not have the above-mentioned disadvantages for the production of a protein, especially for the production of a glycoprotein, and which yields high protein titres.

This object has been achieved with a novel glutamine-auxotrophic human cell according to claim 1 and with a novel process according to claim 7.

According to the invention a glutamine-auxotrophic human cell is provided which has been transfected with a (first) exogenous DNA sequence encoding a protein or an exogenous DNA sequence capable of altering the expression of an endogenous gene encoding a protein, and further with a (second) exogenous DNA sequence encoding a glutanine synthetase (GS), preferably a mammalian GS, wherein these exogenous DNA sequences are located on one or more than one DNA construct, said transfected cell capable of producing said protein and capable of growing in a glutamine-free medium.

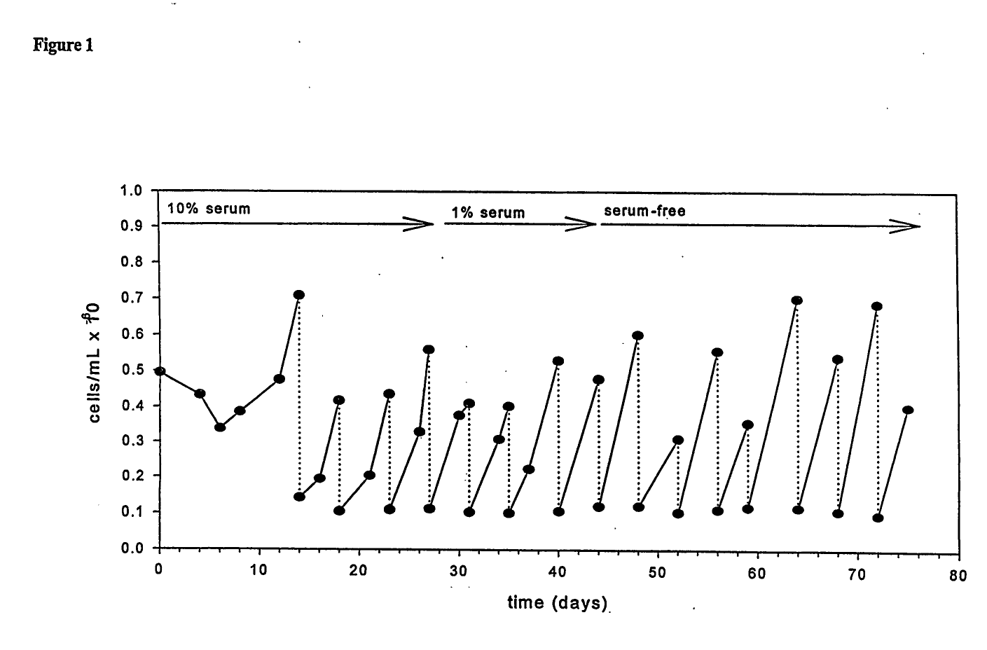

FIG. 1 shows adaptation of cell line R223 to suspension culture in serum-free medium—profiles of cell concentration during repeated serial subculture.

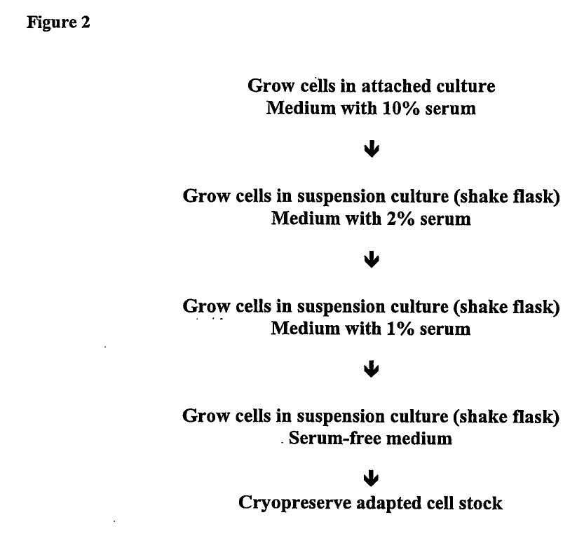

FIG. 2 shows schematic for adaptation of HT1080 cells to suspension culture in serum-free medium.

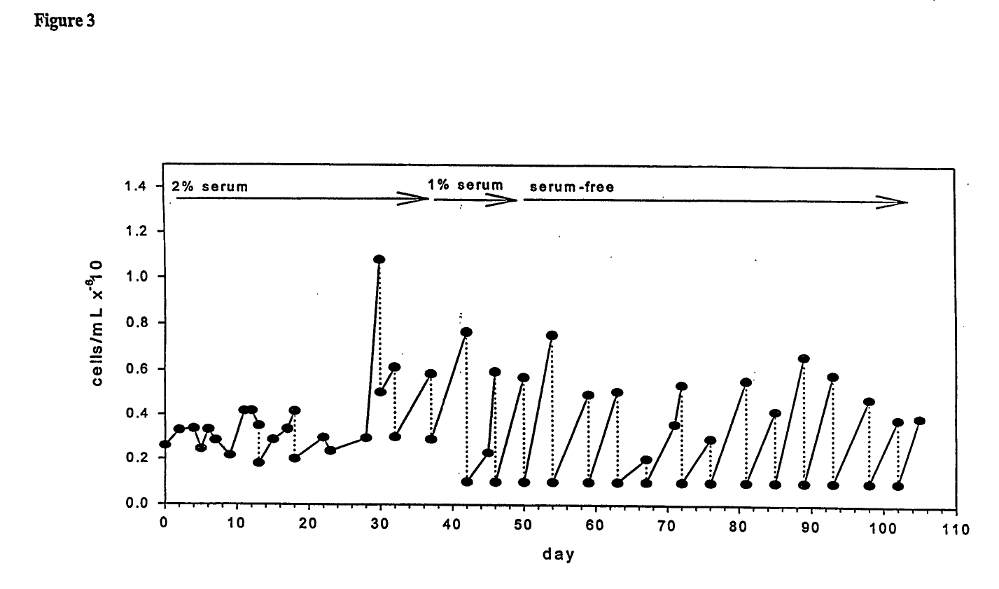

FIG. 3 shows adaptation of cell line HT1080 to suspension culture in serum-free medium—profiles of cell concentration during repeated serial subculture.

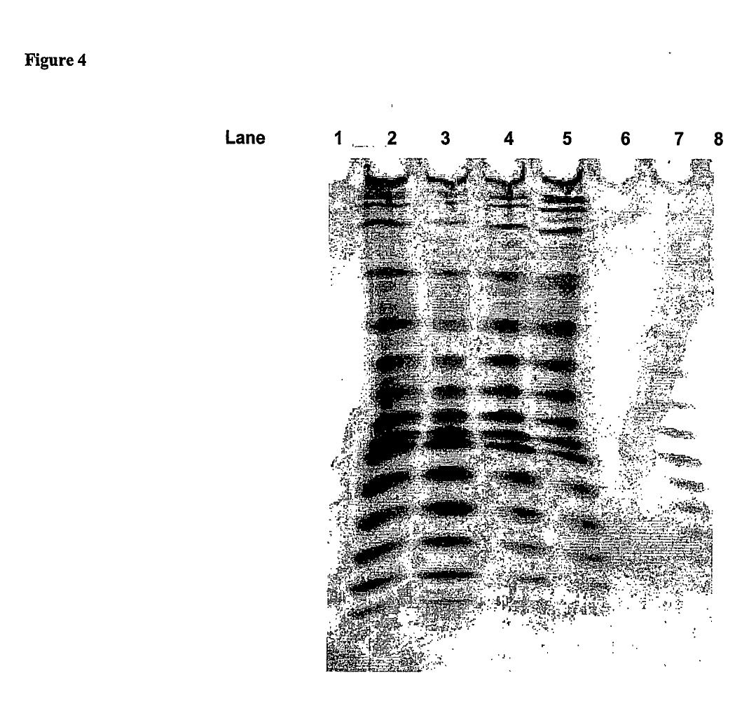

FIG. 4 shows IEF analysis of immunopurified EPO from GS transfectant 3E10 and from the non-transfected R223 cell line grown in industrial high-density growth culture medium. Lane 2: 3E10 harvest. Lane 3:3E10 peak. Lane 4: non-transfected R223 cell line peak. Lane 5: non-transfected R223 cell line harvest.

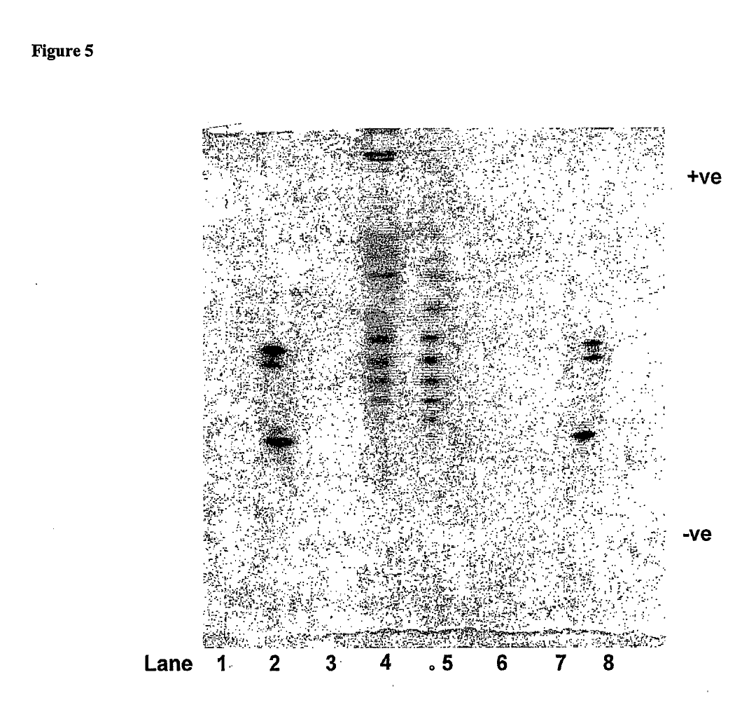

FIG. 5 shows IEF analysis of immunopurified EPO from GS-R223 transfectant 3E10 of the R223 cell line and from the non-transfected R223 cell line grown in conventional Iscove's medium. Lane 4: non-transfected R223 cell line harvest in glutamine-supplemented Iscove's. Lane 5: GS-223 cell line 3E10 at harvest in glutamine-free Iscove's.

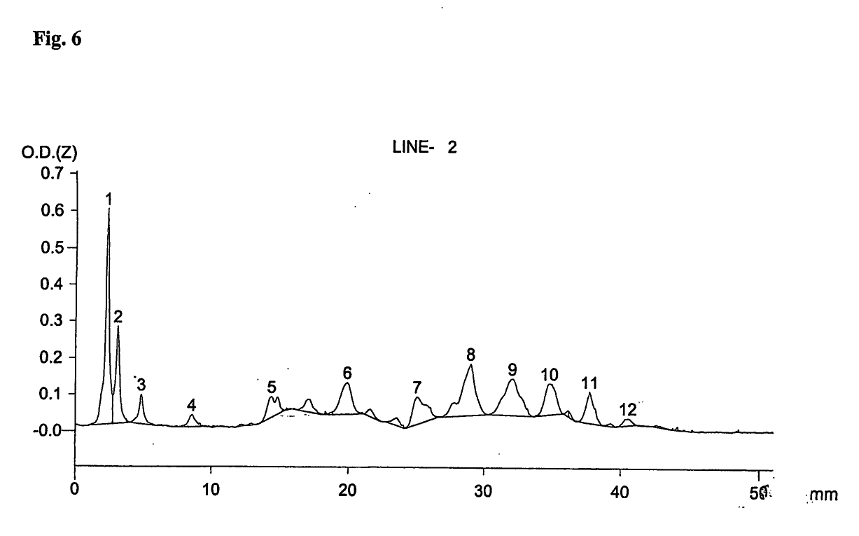

FIG. 6 shows the chromatogram of a densiometric scan of gel lane 4 from top to bottom as shown in FIG. 5

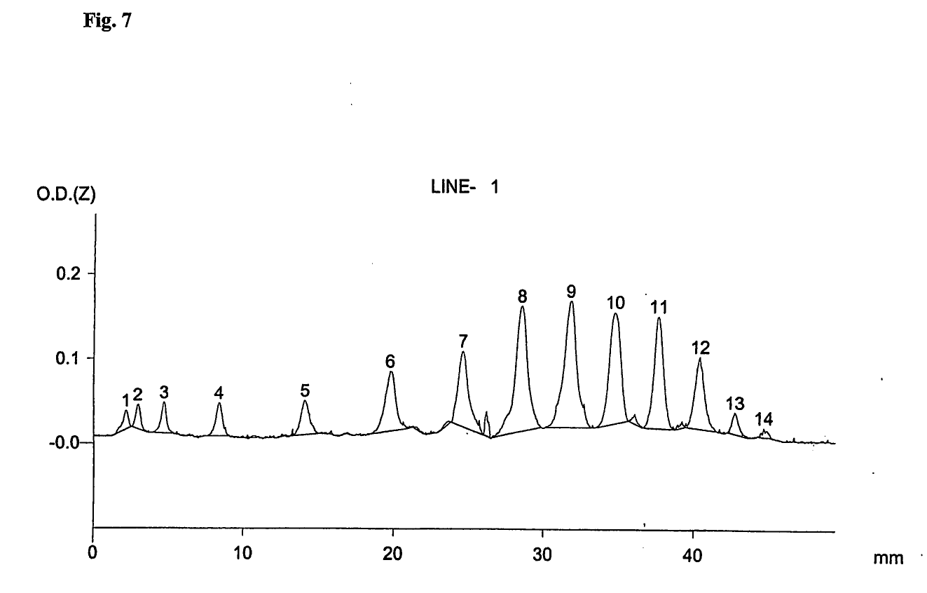

FIG. 7 shows the chromatogram of a densiometric scan of gel lane 5 from top to bottom as shown in FIG. 5

An “exogenous DNA sequence encoding a protein” or an “exogenous DNA sequence capable of altering the expression of an endogenous gene encoding a protein” and an “exogenous DNA sequence encoding a GS” are usually located on a DNA construct like an expression vector or an infectious vector. As expression vector a plasmid can be employed. As infectious vectors can be employed e.g. retroviral, herpes, adenovirus, adenovirus-associated, mumps and poliovirus vector. Preferably an expression vector, in particular a plasmid is used.

An “exogenous DNA sequence encoding a protein” may include additional sequences such as a regulatory sequence as e.g. a promoter and/or an enhancer, polyadenylation sites and splice junctions usually employed for the expression of the exogenous gene or may include additionally one or more separate targeting sequences and optionally DNA encoding a selectable marker as described in WO 93/09222.

An “exogenous DNA sequence capable of altering the expression of the endogenous gene encoding a protein” may include exogenous DNA sequences which do not encode a gene product of the protein but encode part of that gene product e.g. an exon, and may include additional sequences such as regulatory sequences and splice junctions usually employed for the expression of the exogenous DNA sequence. They may further include targeting sequences and optionally DNA encoding a selectable marker as described in WO 93/09222.

Usually, an “exogenous DNA sequence capable of altering the expression of an endogenous gene encoding a protein” is inserted into chromosomal DNA of the cell after transfection into the cell. Homologous recombination or targeting is hereby used to replace or disable the regulatory region normally associated with the endogenous gene with a regulatory sequence. As regulatory sequence may serve e.g. a promoter and/or an enhancer which causes the gene to be expressed at levels higher than evident in the corresponding nontransfected cell as described e.g. in WO 93/09222. Appropriate promoters can be regulatable or constitutively expressed promoters. Appropriate promoters may be strong promoters which are depending on the cell line used e.g. the human cytomegalovirus major immediate early promoter (hCMV-MIE), SV 40 early and late promoters, other promoters of the adenoviruses, early and late promoters of any of the polyoma viruses or papova-viruses, interferon α1 promoter, mouse metallothionein promoter, the rous sarcoma virus long terminal repeat promoter, β-globin promoter, conalbumin promoter, ovalbumin promoter, mouse β-globin promoter and human β-globin promoter.

The “exogenous DNA sequence encoding a GS” according to the present invention might be under the control of a strong promoter as well as under the control of a weak promoter. A strong promoter is used if the exogenous DNA sequence is required simply to express the gene encoding GS. A weak promoter is used if the exogenous DNA sequence is being used as a selectable marker and if GS is used for amplification. An appropriate promoter can be a regulatable or a constitutively expressed promoter. The promoter might be selected such as, that the GS is expressed at a concentration sufficient for growth of the transfected cell but which does not produce a high level of the glutamine catabolite product ammonia in cell culture, usually not more than 4 mM, preferably not more than 2 mM, more preferably less than 2 mM ammonia.

A “selectable marker” confers a selectable phenotype which makes it possible to identify and isolate recipient cells. GS can be used as the selectable marker in the present invention in order to select successfully transfected glutamine-auxotrophic human cells which have incorporated and express the exogenous DNA sequence encoding GS.

A strong promoter may, depending on the cell line used, be e.g., hCMV-MIE, SV 40 early and late promoters, other promoters of the adenoviruses, early and late promoters of any of the polyoma viruses or papova-viruses, interferon al promoter, mouse metallothionein promoter, the rous sarcoma virus long terminal repeat promoter, β-globin promoter, conalbumin promoter, ovalbumin promoter mouse β-globin promoter and human β-globin promoter.

A weak promoter may, depending on the cell line used, be e.g. murine leukaemia virus long terminal repeat, herpes simplex virus thymidine kinase and Mouse Mammary Tumor Virus-Long Terminal Repeat. Preferably the gene encoding a GS is under the control of a strong promoter, more preferably under the control of the hCMV-MIE promoter. A possible embodiment, an amplifiable, mammalian GS sequence from hamster and its use as a selectable marker in mammalian cells is well known in the art and is e.g. described in WO 87/04462, WO 91/06657 and WO 89/01036; the examples of the present invention employ such hamster GS expression unit and respective selection methods as set forth in the references.

The “exogenous DNA sequence encoding a protein” or the “exogenous DNA sequence capable of altering the expression of an endogenous gene encoding a protein” and the “exogenous DNA sequence encoding a GS” are located on one or more than one DNA construct. Preferably, these exogenous DNA sequences are located on more than one, more preferably on two DNA constructs. If these exogenous DNA sequences are located on one DNA construct they might be functionally combined, e.g. in that their expression is driven by the same regulatory sequence e.g. promoter and/or enhancer as described e.g. in WO 89/10404.

Glutamine-auxotrophic human cells means all human cells which do not express GS or express GS poorly, thus being capable of growth in a culture medium containing glutamine but failing to grow or growing only poorly in glutamine-free medium. Glutamine-auxotrophic human cells which are used in the present invention are mortal glutamine-auxotrophic human cells or immortalized glutamine-auxotrophic human cells. Mortal glutamine-auxotrophic human cells are glutamine-auxotrophic human cells which exhibit a limited lifespan in culture. Immortalized, also called “permanent” or “established”, glutamine-auxotrophic human cells are glutamine-auxotrophic cells which exhibit an apparently unlimited lifespan in culture when duly passaged and subcultured as is well-known to those in the art.

Examples of mortal glutamine-auxotrophic human cells may be human fibroblasts and human foetal lung tissue cells. Examples of immortalized glutamine-auxotrophic human cells may be human fibrosarcoma cells, like a HT1080 cell line (e.g. DSMZ No. ACC-315 or ATCC No. CCL 121) and B-lymphoblastoid human cells like a HL60 (DSMZ No. Acc-3). Preferably used in the present invention are immortalized glutamine-auxotrophic human cells. Further preferred, such immortalized glutamine-auxotrophic human cells are B-lymphoblastoid cells or fibrosarcoma cells, more preferably human fibrosarcoma cells, most preferably a HT1080 cell line (e.g. ATCC No. CCL 121) is used.

The glutamine-auxotrophic human cell can be transfected with the exogenous DNA sequences by known genetic engineering techniques.

Transfection with the exogenous DNA sequences depends on whether the sequences are located on one or more than one DNA construct. If the sequences are located on more than one DNA construct, transfection can occur with each sequence separately or by co-transfection. In case transfection occurs with each sequence separately the order of transfection of the sequences is usually optional. Transfection with each sequence separately occurs preferably firstly with the “exogenous DNA sequence encoding said protein” or the “exogenous DNA sequence capable of altering the expression of an endogenous gene encoding said protein” and secondly with the “exogenous DNA sequence encoding a GS”. The transfected glutamine-auxotrophic cell might be cultured after each separate transfection and assessed for protein production.

In order to select for successfully transfected cells, these are grown in a glutamine-free medium. Cells might be grown directly in a glutamine-free medium or at first in a medium containing glutamine which will be diluted stepwise to a glutamine-free medium, e.g. one may start with a glutamine concentration of 10 mM which may be diluted by steps of 2 mM to 0 mM. The appropriate selection procedure might be chosen depending on the cell lines used. As evident from the aforesaid, the glutamine-auxotrophic human cell of the present invention capable of producing a protein and capable of growing in a glutamine free medium is obtainable by transfecting said cell with an exogenous DNA sequence encoding said protein or an exogenous DNA sequence capable of altering the expression of an endogenous gene encoding said protein and an exogenous DNA sequence encoding a glutamine synthetase, wherein these exogenous DNA sequences are located on one or more than one DNA construct.

The exogenous DNA sequence encoding a protein or the exogenous sequence capable of altering the expression of an endogenous gene encoding a protein may be amplified after transfection according to known methods in gene amplification as described in e.g. WO 94/12650. Amplifiable genes encoding enzymes like e.g. DHFR (dihydrofolate reductase), GS, adenosine deaminase, asparagine synthetase, aspartate transcarbamylase, metallothionein-1, ornithine decarboxylase, P-glycoprotein, ribonucleotide reductase, thymidine kinase or xanthine-guanine phosphoribosyl transferase can be used for this purpose. Cells containing amplified copies of these genes are e.g. capable of surviving treatment in media lacking the metabolic product of the enzymes or in media containing a corresponding selective agent. Corresponding selective agents are e.g. methotrexate (MTX) in case of DHFR and methionine sulphoximine (MSX) in the case of GS.

Proteins which are produced by the transfected glutamine-auxotrophic human cell of the present invention are non-glycosylated and glycosylated proteins. Glycosylated proteins refer to proteins having at least one oligosaccharide chain.

Examples for non-glycosylated proteins are e.g. non-glycosylated hormones like luteinizing hormone-releasing hormone, thyroid hormone-releasing hormone, insulin, somatostatin, prolactin, adrenocorticotropic hormone, melanocyte-stimulating hormone, vasopressin, and derivatives thereof e.g., desmopressin, oxytocin, calcitonin, parathyroid hormone (PTH) or fragment thereof (e.g. PTH (I-43)), gastrin, secretin, pancreozymin, cholecystokinin, angiotensin, human placental lactogen, human chorionic gonadotropin (HCG), caerulein and motilin; non-glycosylated analgesic substances like enkephalin and derivatives thereof (see U.S. Pat. No. 4,277,394 and EP-A 031567), endorphin, daynorphin and kyotorphin; non-glycosylated enzymes like non-glycosylated nerve transmitters e.g. bombesin, neurotensin, bradykinin and substance P; non-glycosylated growth factors of the nerve growth factor (NGF) family, of the epithelial growth factor (EGF) and of the fibroblast growth factor (FGF) family and non-glycosylated receptors for hormones and growth factors.

Examples for glycosylated proteins are hormones and hormone releasing factors like growth hormones, including human growth hormone, bovine growth hormone, growth hormone releasing factor, parathyroid hormone, thyroid stimulating hormone, EPO, lipoproteins, alpha-1-antitrypsin, follicle stimulating hormone, calcitonin, luteinizing hormone, glucagon, clotting factors such as factor VIIIC, factor IX, tissue factor, and von Willebrands factor, anti-clotting factors such as Protein C, atrial natriuretic factor, lung surfactant, a plasminogen activator such as urokinase or human urine or tissue-type plasminogen activator (t-PA), thrombin, hemopoietic growth factor, enkephalinase, RANTES (regulated on activation normally T-cell expressed and secreted), human macrophage inflammatory protein (MIP-1-alpha), a serum albumin such as human serum albumin, mullerian-inhibiting substance, relaxin A-chain, relaxin B-chain, prorelaxin, mouse gonadotropin-associated peptide, a microbial protein, such as beta-lactanase, DNase, inhibin, activin, renin, vascular endothelial growth factor (VEGF), receptors for hormones or growth factors, integrin, protein A or D, rheumatoid factors, a neurotrophic factor such as bone-derived neurotrophic factor (BDNF), neurotrophin-3, -4, -5 or -6 (NT-3, NT-4, NT-5 or NT-6), or a nerve growth factor such as NGF-β, platelet-derived growth factor (PDGF), fibroblast growth factor such as a FGF and bFGF, epidermal growth factor (EGF), transforming growth factor (TGF) such as TGF-alpha and TGF-beta, including TGF-β1, TGF-β2, TGF-β3, TGF-β4 or TGF-β5, insulin-like growth factor-I and -II (IGF-I and IGF-II), des(1-3)-IGF-I (brain IGF-I), insulin-like growth factor binding proteins, CD proteins (cluster of differentiation proteins) such as CD-3, CD4, CD-8 and CD-19, osteoinductive factors, immunotoxins, a bone morphogenetic protein (BMP), cytokines and their receptors, as well as chimeric proteins comprising cytokines of their receptors, including, for instance tumor necrosis factor alpha and beta, their receptors (TNFR-1, EP 417 563, and TNFR-2, EP 417 014) and their derivatives, an interferon such as interferon-alpha, -beta and -gamma, colony stimulating factors (CSFs), e.g., M-CSF, GM-CSF and G-CSF, interleukins (ILs), e.g., IL-1 to IL-10, superoxide dismutase, T-cell receptors, surface membrane proteins, decay accelerating factor, viral antigen such as, for example, a portion of the AIDS envelope, transport proteins, homing receptors, addressins, regulatory proteins, antibodies, chimeric proteins, such as immunoadhesins, and fragments of any of the above listed glycosylated proteins. Preferably, glycosylated proteins are produced in the present invention. More preferably N-glycosylated proteins are produced in the present invention. Most preferably glycosylated hormones like EPO which is N-glycosylated and whose bioactivity is dependent thereon, or in particular EPO are produced.

Any suitable culture procedure and culture apparatus known in the art may be used for growing the transfected human cell of the present invention. As culture medium common glutamine-free basal medium supplemented with about 0.1 to 20%, preferably 0.5 to 15% serum as well as serum-free, glutamine-free common basal medium can be used. Also, common glutamine-free basal medium free of protein of animal origin can be used. Preferably serum-free glutamine-free common basal medium is used.

Sera that may be used are e.g. foetal bovine serum or adult bovine serum. Preferably foetal bovine serum is used. Common glutamine-free basal medium that may be used are e.g. glutamine-free Eagle's minimal essential medium (MEM) medium, glutamine-free Dulbecco's Modification of Eagle's Medium (DMEM), glutamine-free Iscove's DMEM medium (N. Iscove and F. Melchers, Journal of Experimental Methods, 1978, 147, 923), glutamine-free Ham's F12 medium (R. G. Ham, Proceedings of National Academy of Science, 1965, 53, 288), glutamine-free L-15 medium (A. Leibovitz, American Journal of Hygiene, 1963, 78, 173), glutamine-free RPMI 1640 medium (G. E. Morre et al., The Journal of the American Medical Association, 1967, 199, 519), a glutamine-free proprietary medium and suitable ratio mixtures thereof. Fortification of common cell culture growth media for high-density cell culture is well-known in the art and is described e.g. in GB 2251249. It is well-applicable to the glutamine-free media of the present invention, too.

Common supplements might be added to the common glutamine-free basal medium. Supplements that might be usually added contain proteins usually present in serum and optionally further ingredients which have a positive effect on cell growth and/or cell viability. Proteins usually present in serum are e.g. bovine serum albumin (BSA), transferrin and/or insulin. Further ingredients which have a positive effect on cell growth and/or cell viability are e.g. soybean lipid, selenium and ethanolamine: Amino acids which replace glutamine and/or nucleosides might be added to the culture medium depending on the cell line. Examples of amino acids are isoleucine, leucine, valine, lysine, asparagine, aspartic acid, glutamic acid, serine, alanine. Optionally glutamine might be added to the common glutamine-free basal medium at low concentrations of usually less than 1 mg/I, preferably less than 0.5 mg/l to support it's biosynthetic function (e.g. transamination reactions).

If the exogenous DNA sequence encoding a protein or the exogenous sequence capable of altering the expression of an endogenous gene encoding a protein has been amplified after transfection using amplifiable genes, the corresponding selective agent maybe added to the common glutamine-free basal medium. The applied concentration range of the selective agent does depend on the cell line used. Usually, concentrations of 10 μM and higher are used.

A glutamine-auxotrophic human cell which cell can be used as starting material for obtaining a transfected glutamine-auxotrophic human cell according to the present invention which transfected cell is capable of producing a protein and further is capable of growing in a glutamine-free medium according to the invention might be anchorage-dependent or anchorage-independent. If an anchorage-dependent human cell, e.g. the HT1080 cell line (ATCC No. CCL 121) is used, it can be adapted to be a anchorage-independent HT1080 cell line capable of growing in suspension in serum-free medium which has not been described in the literature yet.

Adaptation might occur before or after transfection with the exogenous DNA sequence encoding a protein, or the exogenous DNA sequence capable of altering the expression of an endogenous gene encoding a protein and the exogenous DNA sequence encoding a GS. Preferably, the cell is firstly transfected with the exogenous DNA sequence encoding a protein or the exogenous DNA sequence capable of altering the expression of an endogenous gene encoding a protein, and secondly adapted for growing in suspension in serum-free medium and then further transfected with the exogenous DNA sequence encoding a GS. If necessary the transfected cell might be again adapted for growing in suspension in serum-free, glutamine-free medium.

The transfected glutamine-auxotrophic human cell of the present invention might be anchorage-dependent or anchorage-independent and might be capable of growing in suspension in serum-free, glutamine-free medium. The preferred transfected glutamine-auxotrophic human cell is anchorage-independent and is capable of growing in suspension in serum-free, glutamine-free medium.

The adaptation for being an anchorage-independent cell capable of growing in suspension in serum-free medium can be achieved by adapting the cell in a first step to be anchorage-independent using serum-containing medium. This can be done by e.g. trypsinisation of the cells and subsequent agitation or releasing the cells by agitation. The cells are then adapted in a second step to serum-free medium by subsequent reduction of serum content. During adaptation the amount of selective agent if used can be reduced in case it is inhibitory to growth of the cells. However, the cells might be as well adapted in a first step to grow in serum-free medium by subsequent reduction of serum content and in a second step adapted to be anchorage-independent cells capable of growing in suspension by e.g. trypsinisation of the cells and subsequent agitation or releasing the cells by agitation. Both steps might be as well applied simultaneously.

Preferably the cells are adapted in a first step to be anchorage-independent cells using serum-containing medium by releasing the cells by agitation and then adapting them in a second step to serum-free medium by subsequent reduction of serum content.

As a basis for serum-containing medium a culture medium as described above can be used. Selective agents as defined herein may be added to the culture medium. The applied concentration range of the selective agent does depend on the cell line used. Usually concentration of 10 μM and higher are used. Serum-containing medium is usually supplemented with about 0.1 to 20% preferably 0.2 to 10% most preferably 0.5 to 5% serum. Sera that may be used are as mentioned above. Subsequent reduction of serum content might be obtained by reducing the serum content stepwise e.g. from 10% to 1% to 0%.

The transfected glutamine-auxotrophic human cell of the present invention is used in a process for the production of a protein by culturing said cell in a culture medium under conditions suitable for expression of said protein and recovering said protein. Proteins produced are as described above. As culture medium common glutamine-free basal media and common supplements as described above can be used. Suitable culture conditions are those conventionally used for in vitro cultivation of mammalian cells as described e.g. in WO 96/39488.

Protein can be isolated from the cell culture by conventional separation techniques such as e.g. fractionation on immunoaffinity or ion-exchange columns; precipitation; reverse phase HPLC; chromatography; chromatofocusing; SDS-PAGE; gel filtration. One skilled in the art will appreciate that purification methods suitable for the polypeptide of interest may require modification to account for changes in the character of the polypeptide upon expression in recombinant cell culture.

### EXAMPLES

### Example 1

### Provision of the Human Fibrosarcoma Cell Line HT1080-R223

The anchorage-dependent human HT1080-R223 cell line containing multiple copies of the human EPO gene is a cell line used in industrial production of EPO and was originally created by Transkaryotic Therapies, Inc. Cambridge, Mass. 02139 (US). It is derived from anchorage-dependent human fibrosarcoma HT 1080 cell line. The parent HT1080 cell line (ATTC No. CCL 121) has acquired the capability of producing EPO by transfection with the DNA construct pREPO22 which is similar to the DNA construct pREPO 18 described in WO 95/31 560 except that the DHFR gene is in the opposite orientation and that pREPO22 does contain approximately 600 base pairs less homologous sequence than pREPO18. This cell line is further referred to as the R223 cell line for short.

### Example 2

### Adaptation of the R223 Cell Line to Growth in Suspension in Serum-Free Medium

Cells grown as attached cultures in static flasks were released by trypsinization, resuspended into a proprietory glutamine-containing serum-free medium further referred to as “HM9” supplemented with 10% dialysed foetal bovine serum (dFBS) and 500 nM MTX and incubated as shake flask cultures. Growth commenced after 6 days and the cells were subcultured into the same medium (FIG. 1). Once a reliable pattern of growth had become established the serum content of the medium was reduced to 1%. Again reliable growth was allowed to become re-established before complete elimination of the serum supplement.

Serum-supplemented and serum-free cultures were overgrown to assess productivity. Results are shown in Table 1 together with data from attached cultures. After adequate adaptation specific growth rate in suspension culture, even in the absence of serum, was equivalent to that in serum-supplemented attached culture.

Upon adaptation from attached culture to suspension culture in serum supplemented medium the specific rate of EPO synthesis decreased by 50%, from 24 to 12 EU/10⁶ cells/h. However, this rate was substantially restored, to 18 EU/10⁶ cells/h, following adaptation to serum-free growth.

TABLE 1
Growth and productivity of the R223 cell line in attached culture and in suspension
culture before and after adaptation to serum-free medium.

| | maximum | specific | cumulative | | |
|---|---|---|---|---|---|
| | cells/ | growth | cell h/ | EPO | q_{EPO} |
| | mL × 10⁻⁶ | rate h⁻¹ | ml × 10⁻⁶ | EU/ml | EU/10⁶ cells/h |
| attached culture | not | 0.0157 | not | not | 24 |
| (DMEM + 10% | applicable | | applicable | applicable | |
| dFBS 500 nM MTX) | | | | | |
| suspension culture | 1.4 | 0.0185 | 334 | 4199 | 12 |
| (HM9 + 10% dFBS | | | | | |
| 500 nM MTX) | | | | | |
| suspension culture | 1.1 | 0.0166 | 210 | 3600 | 18 |
| (HM9 (serum-free) | | | | | |
| 500 nM MTX) | | | | | |

### Example 3

### Adaptation of the HT1080 (ATCC CCL121) to Growth in Suspension in Serum-Free Medium

The HT1080 cell line ATCC CCL121 was obtained from the American Type Culture Collection (Rockville, Md., USA). Initially cells were grown in attached cultures in DMEM containing 10% foetal bovine serum (FBS).

The steps to suspension and serum-free adaptation can be performed according to the schematic represented in FIG. 2. To initiate suspension cultures, cells were released from attached cultures by trypsinization and the released cells were resuspended into HM9 plus 2% FBS. These were then incubated as shaken cultures. Once suspension growth of the cells in HM9 plus 2% FBS had become established the cultures were diluted with the same medium to make daughter cultures. Reliable suspension growth of the HT1080 cell line was established after 30 days in culture (FIG. 3). Thereafter the serum content of the medium was reduced, and finally eliminated completely. The cells continued to grow in serial subculture in the absence of serum.

### Example 4

### Effect of Ammonia on Growth and Productivity of the R223 Cell Line

Ammonia is a catabolite produced by cultured cells when glutamine is used as an energy substrate. It is cytotoxic and can inhibit cell growth. Furthermore, it can inhibit glycosylation of proteins by its effect on pH within the Golgi of the cell. R223 cells in flask cultures typically produce 5 mM ammonia in unfed cultures and 10 mM in fermenter cultures which receive a nutrient feed.

In an initial examination of the effects of ammonia, R223 cells were grown in shake flasks, either without added ammonia, or with ammonia added at 2, 5 or 10 mM. For each concentration of ammonia replicate cultures were set up at three different pH values, by varying the CO₂ content of the overlay gas. The primary aim here was to determine the extent of growth inhibition exerted by ammonia and to test whether reduced pH would overcome this growth inhibition.

While ammonia was found to inhibit cell growth (Table 2), reduced pH failed to alleviate the inhibitory effects of ammonia, though the elevated pCO₂ required to reduce the pH may itself have caused some growth inhibition.

Cultures were terminated after only 3 days. To continue any longer was not valid because accumulation of non-volatile acid catabolites caused the pH of all cultures to fall by several points of a pH unit.

TABLE 2
Effect of Ammonia on growth and productivity of R223 cells at
different values of culture pH. The pH was adjusted by the
content of CO₂ in the overlay gas.

| | ammonia | | maximum | specific | | q_{-EPO} |
|---|---|---|---|---|---|---|
| pCO₂ | added | initial | cells/ | growth rate | EPO | EU/10⁶ |
| % | mM | pH | ml × 10⁻⁶ | h⁻¹ | EU/ml | cells/h |
| 5 | 0 | 7.26 | 0.9 | 0.020 | 1719 | 35 |
| 10 | 0 | 6.93 | 0.7 | 0.017 | 1580 | 40 |
| 15 | 0 | 6.75 | 0.4 | 0.012 | 1186 | 56 |
| 5 | 2 | 7.15 | 0.8 | 0.018 | 1796 | 38 |
| 10 | 2 | 6.88 | 0.7 | 0.017 | 1928 | 42 |
| 15 | 2 | 6.80 | 0.3 | 0.009 | 1404 | 78 |
| 5 | 5 | 7.18 | 0.6 | 0.018 | 2639 | 40 |
| 10 | 5 | 6.91 | 0.6 | 0.014 | 1663 | 51 |
| 15 | 5 | 6.75 | 0.3 | 0.016 | 1626 | 45 |
| 5 | 10 | 7.17 | 0.5 | 0.016 | 2222 | 43 |
| 10 | 10 | 6.90 | 0.6 | 0.014 | 1966 | 50 |
| 15 | 10 | 6.75 | 0.2 | 0.008 | 1891 | 86 |

In a subsequent experiment (Table 3) pH was varied by adjusting the NaHCO₃ content of the medium while maintaining the CO₂ content of the overlay gas at a non-inhibitory concentration. The range of pH tested was from pH 7.0 to 7.5. (It must be emphasized that in flask cultures medium pH cannot be controlled and falls substantially, even over the first two days of culture. The pH values stated are the initial pH of the each culture). Specific growth rate was maximal at the pH 7.5 (NaHCO₃ at 3 g/l) but at this pH both growth rate and maximum cell concentration were halved by the presence of 10 mM ammonia while the specific rate of EPO synthesis was reduced four fold (Table 3).

At pH 7.25 (NaHCO₃ at 1.5 g/l) specific growth rate was reduced, and the specific rate of EPO synthesis was increased, compared to the culture at pH 7.5. However growth rate was less affected, and the specific rate of EPO synthesis was unaffected, by the presence of 10 mM ammonia.

At the lowest pH studied, pH 7.0 (NaHCO₃ at 0.75 g/l) specific growth rate was further reduced but again was less affected by ammonia than at pH 7.5. The specific rate of EPO synthesis was increased in the presence of added ammonia (this may have been attributable to the reduction in growth rate).

TABLE 3
Effect of ammonia on growth and productivity of R223 cells at
different values of culture pH. The pH was adjusted by varying
the content of NaHCO₃ in the medium.

| | | maximum | specific | | q_{EPO} |
|---|---|---|---|---|---|
| | initial | cells/ | growth rate | EPO | EU/10⁶ |
| | pH | ml × 10⁻⁶ | h⁻¹ | EU/ml | cells/h |
| NaHCO₃ 3 g/l | 7.49 | 1.3 | 0.030 | 951 | 16 |
| NaHCO₃ 3 g/l | 7.51 | 0.5 | 0.016 | 256 | 4 |
| 10 mM ammonia | | | | | |
| NaHCO₃ 1.5 g/l | 7.26 | 1.1 | 0.027 | 1151 | 24 |
| NaHCO₃ 1.5 g/l | 7.27 | 0.7 | 0.021 | 1191 | 26 |
| 10 mM ammonia | | | | | |
| NaHCO₃ 0.75 g/l | 7.02 | 0.9 | 0.020 | 995 | 26 |
| NaHCO₃ 0.75 g/l | 7.01 | 0.4 | 0.016 | 1009 | 42 |
| 10 mM ammonia | | | | | |

### Example 5

### Productivity of the R223 Cell Line in Fermenter Cultures at 5 Litre Scale

For the R223 cell line three 5 litre fermentations were carried out, controlled at different values of pH between 6.95 and 7.15 (see Table 4). Each culture received a concentrated nutrient feed containing amino acids and glucose designed to maintain major consumed nutrients at sufficient concentration. Cultures grew well at pH 7.15 and at pH 7.05, but the culture at pH 6.95 failed to grow, probably due to the high concentration of CO₂ required to control at that pH.

TABLE 4
Productivity and metabolism of the R223 cell line in fermenter culture.

| | | | cumulative | | | |
|---|---|---|---|---|---|---|
| | maximum | doubling | cell | | q_{EPO} | Final |
| fermentation | cells/ | time | hours/ | EPO | EU/10⁶ | Ammonia |
| conditions | ml × 10⁻⁶ | hours | ml × 10⁻⁶ | EU/ml | cells/h | mg/l |
| pH 7.15 | 1.0 | 46 | 197 | 10795 | 58 | 163 |
| NaHCO₃ at 2 g/l | | | | | | |
| pH 7.05 | 1.0 | 51 | 195 | 11414 | 64 | 157 |
| NaHCO₃ at 2 g/l | | | | | | |
| pH 6.95 | 0.2 | 105 | 62 | 2995 | 70 | not done |
| NaHCO₃ at 2 g/l | | | | | | |

### Example 6

### Transfection of R223 with GS and Productivity of GS Transfectants in Attached Culture

Cells used for the transfection were from a suspension-adapted serum-free stock of the R223 cell line of example 2. As these cells grow as large multicellular aggregates they were trypsinised to reduce them to substantially single-cell suspension.

For transfection an aliquot of approximately 10⁷ of the single-suspension adapted R223 cell line (in phosphate buffered saline without calcium or magnesium) obtained in example 2 was mixed with 20 μg of linearized DNA containing the GS gene (DNA sequence pCMGS Bam H1 described in Bebbington et al., 1992, Biotechnology 10, 169-175) and subjected to electroporation using a Biorad Gene Pulser (450 volts, 250 μF). As a control an equivalent aliquot of cells was electroporated without addition of DNA.

Cells were diluted with HM9 medium without MTX containing 0 or 10% dFBS and distributed into 96-well culture plates or into 25 cm² flasks and incubated at 35.5 to 37° C. HM9 medium initially contained 2 mM glutamine but this was diluted to 0.5 mM after 1 day and then replaced after ten days with glutamine-free HM9 medium. Also at day 1 or day 10, MTX (500 nM) was re-introduced to the cultures.

For the cultures set up with the control cells, which were electroporated without added DNA, no growth was obtained.

For the cells which were electorporated with DNA, 15 GS transfectants were identified in six 96-well plates. GS transfectants were obtained both with, and without, MTX in the initial medium. Of the 15 GS transfectants seven were successfully expanded to flask cultures (Table 5). The remaining eight GS transfectants exhibited aberrant cell morphology, or grew poorly, and were abandoned.

For each of the initial seven GS transfectants isolated, a set of replicate static flask cultures was set up using 10% serum-supplemented glutamine-free HM9 medium. At intervals of 1 to 4 days, cultures were sacrified in order to count the cells and to measure the concentration of EPO by EPO-ELISA. From a composite of these data, the specific rate of EPO synthesis could be estimated for each GS transfectants. Data are summarized in Table 5 and, for comparison, data for the non-transfected R223 cell line are included. All GS transfectants exhibited elevated specific rates of EPO synthesis compared to the non-transfected R223 cell line. The best GS transfectant, 3E10, had a synthesis rate five to six fold higher than the non-transfected R223 cell line.

TABLE 5
Specific rates of EPO synthesis for GS transfectants of the R223 cell
line and for the non-transfected R223 cell line. Data are derived
from attached cultures grown in the presence of 10% dFBS.
Methotrexate was omitted from the medium used for making the
transfectants, but was reintroduced to the cultures at day 1 or 10
after transfection.

| | day of addition of | |
|---|---|---|
| | MTX (500 nM) to | q_{EPO} (attached) |
| GS transfectant | transfection plate | EU/10⁶ cells/h |
| 3B3 | 10 | 59 |
| 3E10 | 10 | 139 |
| 3E11 | 10 | 45 |
| 4D9 | 10 | 35 |
| 8F11 | 1 | 47 |
| 8F12 | 1 | 79 |
| 8G3 | 1 | 52 |
| non-transfected R223 cell line | not applicable | 24 |
| assessed in attached culture | | |

### Example 7

### Adaptation of GS Transfectants to Suspension Culture and Serum-Free Medium

Of the seven GS transfectants obtained in Example 6, GS transfectant #3E10 and GS transfectant #8G3 were progressed into suspension culture.

Cells were grown as attached cultures in static flasks in glutamine-free HM9 medium supplemented with 10% dFBS and 500 nM MTX at 35.5 to 37° C. The cells were released after 5 or 6 days by agitation, resuspended into the same medium, and incubated as shake flask cultures at the same temperature. Growth commenced after only two or three days. Cells were subcultured once in the same medium and then overgrown to assess productivity. Titres and product synthesis rate were at least two fold higher than for the non-transfected R223 cell line grown in 10% sFBS and 500 nM MTX (Table 6).

TABLE 6
Growth and productivity of GS transfectants 3E10 and 8G3 in
suspension culture in shake flasks. Glutamine-free HM9 medium
contained 10% dFBS and 500 nM MTX.

| | | | q_{-EPO} |
|---|---|---|---|
| | Max. cells/ | Max. EPO titre | (suspension) |
| GS transfectant | ml × 10⁻⁶ | EU/ml | EU/10⁶ cells/h |
| 3E10 | 0.7 | 11113 | 57 |
| 8G3 | 1.2 | 10171 | 33 |
| non-transfected R223 | 1.4 | 4199 | 12 |
| cell line assessed | | | |
| in suspension culture | | | |
| grown in 10% dFBS | | | |
| and 500 nM MTX | | | |

For suspension cultures of both GS transfectants 3E10 and 8G3 the dFBS content of the glutamine-free HM9 medium was reduced stepwise from 10% to 2% to 1% to 0.2% to 0.1% to 0% for 3E10 and 10% to 2% to 1% for 8G3, allowing reliable cell growth to become established at each dFBS concentration before further reduction. 8-G3 was not further adapted than to 1% dFBS.

### Example 8

### Productivity and Metabolism of GS Transfectant 3E10 in Serum-Free Suspension Culture

The GS transfectant 3E10 adapted to serum-free growth in Example 7 was cultured in suspension culture in serum-free glutamine-free HM9 medium at 35.5 to 37° C. For the GS transfectant 3E10 cell line 2 to 3 fold higher specific rates of EPO synthesis were obtained than for the non-transfected R223 cell line, which was cultured under the same conditions in HM9 medium containing glutamine (Table 7).

TABLE 7
Growth and productivity of GS transfectant 3E10 cells in serum-free
suspension culture.

| | maximum cells/ | EPO | q_{-EPO} |
|---|---|---|---|
| | ml × 10⁻⁶ | EU/ml | EU/10⁶/h |
| 3E10 | 1.1-1.2 | 9731-14234 | 40-67 |
| (glutamine-free HM9 | | | |
| medium) | | | |
| R223 | 1.0-1.6 | 3075-5818 | 13-24 |
| (HM9 medium with | | | |
| 6 mM glutamine) | | | |

The higher EPO-productivity of the GS transfected cell was accompanied by the reduction in the release of metabolic ammonia. Instead of the 5 mM ammonia typically produced in flask cultures of the non-transfected R223 cell line in HM9 medium containing 6 mM glutamine, the GS transfectant 3E10 produced only 1.8 mM ammonia in glutamine-free HM9 medium (Table 8).

TABLE 8
Reduction in ammonia synthesis in GS transfectant 3E10.

| | | | q_{Ammonia} |
|---|---|---|---|
| | Max. cells/ | Ammonia | μMoles/ |
| | ml × 10⁻⁶ | mM | 10⁶ cells/h |
| R223 (HM9 medium containing | 0.9 | 4.8 | 30 |
| 6 mM glutamine) | 1.2 | 5.0 | 23 |
| | 1.0 | 5.5 | 27 |
| 3E10 (glutamine-free HM9 | 0.8 | 1.8 | 11 |
| medium) | | | |

### Example 9

### Analysis of Product Quality

EPO produced by GS transfectant 3E10 in flask cultures has been immunopurified and analysed for its distribution of glycoforms. FIG. 4 shows isolectric focusing (IEF) gel analysis of EPO obtained at peak cell concentration and harvest of a culture of 3E10 cells grown in glutamine-free HM9 medium as described in Example 8. Comparable samples from the non-transfected R223 cell line grown in HM9 medium are included. EPO produced from GS transfectant 3E10 exhibited intensification of the more acidic isoforms compared to the non-transfected R223 cell line. Scanning of the IEF gels has allowed quantitation of the isoform distribution and calculation of a theoretical isoform relative activity (IRA).

TABLE 9
Analysis of isoform relative activity for EPO from non-transfected R223 cell line and
GS transfectant 3E10.

| | | | non-transfected | | GS transfectant | |
|---|---|---|---|---|---|---|
| | | | R223 | | 3E10 | |
| | | | | Activity | | Activity |
| | | | Percentage | (percent × | Percentage | (percent × |
| | Band | Specific | of each | specific | of each | specific |
| | number | Activity* | isoform | activity) | isoform | activity) |
| more basic | 1 | | 5.2 | | 2.5 | |
| | 2 | | 5.2 | | 3.2 | |
| | 3 | | 6.3 | | 3.6 | |
| | 4 | | 11.4 | | 6.0 | |
| | 5 | | 10.5 | | 6.2 | |
| | 6 | 0.071 | 9.2 | 0.7 | 7.5 | 0.5 |
| | 7 | 0.194 | 8.8 | 1.7 | 10.5 | 2.0 |
| | 8 | 0.273 | 8.7 | 2.4 | 11.6 | 3.2 |
| | 9 | 0.373 | 5.5 | 2.1 | 9.3 | 3.5 |
| | 10 | 0.658 | 4.4 | 2.9 | 9.5 | 6.3 |
| | 11 | 0.989 | 4.3 | 4.3 | 7.8 | 7.7 |
| | 12 | 0.999 | 4.5 | 4.5 | 6.1 | 6.1 |
| | 13 | 1.000 | 1.4 | 1.4 | 5.3 | 5.3 |
| | 14 | 0.796 | 0.2 | 0.2 | 1.7 | 1.4 |
| | 15 | | | | 0.5 | |
| | 16 | | | | | |
| more acidic | 17 | | | | | |
| | other minor | | 14.4 | | 8.7 | |
| | bands | | | | | |
| | | | Sum = | Sum = | Sum = | Sum = |
| | | | 100% | IRA = 20.2 | 100% | IRA = 36.0 |

*Values for activity derived from EP-A 0 428 267

The data indicated considerable enhancement of the product quality in the GS transfectant 3E10 (Tables 9 and 10), with an IRA almost two fold higher than for the control culture of the non-transfected R223 cell line.

Also the hypothetical N-glycan charge “Z” was determined for the EPO from GS transfectant 3E10. “Z” was not determined for the non-transfected R223 cell line in Table 10. Nevertheless the “Z” values for GS transfectant 3E10 (273 at peak cell concentration and 265 at harvest) exceed values obtained in flask culture (183-228) for the non-transfected R223 cell line. “Z” was determined according to Hermentin et al., Glycobiology, 1996, 6, 217-230; Z is obtained by multiplying the respective %-share of a certain sialyated isoform with the corresponding negative charge of said isoform, depending on whether it is asialo/neutral, monosialo, tri-, tetra or pentasialo. The mathematical sum of said product terms is Z. The Z-number correlates with the in vivo clearance rate of a given therapeutic glycoprotein (Hermentin, supra).

TABLE 10
Analysis of product quality for EPO from non-transfected R223
cell line and GS transfectant 3E10.

| | | | Isoform | |
|---|---|---|---|---|
| | | titre | relative | |
| | | EU/mL | activity | ‘Z’ |
| R223 parent (HM9 medium | peak | 3518 | 25.2 | not done |
| with 6 mM glutamine) | harvest | 6150 | 20.2 | not done |
| 3E10 (glutamine-free | peak | 3921 | 43.6 | 273 |
| HM9 medium) | harvest | 6862 | 36.0 | 265 |

### Example 10

### Productivity, Metabolism and Product Quality of GS Transfectant 3E10 in Serum-Free Suspension Culture in 6 Litre Fermenter

The GS-transfectant 3E10 was grown in batch culture in airlift fermenters. The culture medium was glutamine-free HM9, without serum. Each culture received a concentrated nutrient feed, containing amino acids and glucose designed to maintain major consumed nutrients at sufficient concentration. Results are shown in Table 11 and data for a fermentation of the non-transfected parent R223 are included for comparison.

The GS transfectant exhibited extended viability, hence an increase in the duration of culture. The specific rate of product synthesis was equivalent to that of the non-transfected parent cell line unchanged, but the greater culture longevity resulted in increased maximum product concentration. Ammonia accumulation was at least four fold lower for the GS transfectant. Product quality, measured by RA, was improved for the GS transfectant.

TABLE 11
Growth and productivity of GS transfectant 3E10 cells in serum-free suspension
culture. Product quality of EPO produced was quantified by IRA.

| | | | | | | q_{EPO} | |
|---|---|---|---|---|---|---|---|
| | Max | Max | | | Harvest | overall | |
| Fermentation | cells/ | CCH*/ | Harvest | Ammonia | Titre | EU/10⁶ | IRA at |
| conditions | ml × 10⁻⁶ | ml × 10⁻⁶ | day | mg/L | EU/ml | cells/h | harvest |
| glutamine-free | 1.03 | 306 | 21 | 41 | 20936 | 69 | 43.6 |
| glutamine-free | 1.13 | 360 | 24 | 20 | 20150 | 52 | 42.6 |
| glutamine-free | 0.94 | 319 | 24 | 15 | 18680 | 49 | 48.5 |
| parent R223 in | 1.11 | 216 | 13 | 165 | 12225 | 58 | 28 |
| standard | | | | | | | |
| process with | | | | | | | |
| glutamine | | | | | | | |

*Cumulative cells hours, the time integral of cell concentration [Renard et al., 1988, Biotechnology Letters 10 (9): 1-96]

### Example 11

### Productivity and Metabolism of GS Transfectant in Serum-Free Suspension Culture in Iscove's-Based Medium

The GS transfected 3E10 adapted to serum-free growth in Example 7 was cultured in shake flask in a serum-free, glutamine-free, version of Iscove's medium. The parent cell line, R223, was grown in parallel in the equivalent medium that had been supplemented with glutamine.

Medium used was Iscove's Modified Dulbecco's Medium with Iscove's supplement (bovine serum albumin at 0.4 g/L, human holo-transferrin at 30 mg/L), recombinant human insulin at 10 mg/L, Lutrol F68 at 1 g/L and ethanolamine at 60 μL/L. The medium contained 4 mM glutamine for culture of the cell line R223, but for the GS transfected cell line the glutamine was omitted and replaced with 4 mM sodium glutamate plus 4 mM asparagine.

The specific rate of product synthesis was approximately 50% higher for the GS transfected cell line, 3E10, than for the non-transfected parent line R223 (Table 12). The specific rate of ammonia production was seven fold lower for the transfected cell line 3E10 (Table 13).

Product was purified from each of these cultures and analysed on isoelectric focusing (IEF) gels. Results of this analysis are shown in FIG. 5 which shows an IEF gel after staining. IEF gel pH 2.5 to 6.5 run under denaturing conditions and stained with Coomassie Blue.

| Key for FIG. 5 | |
|---|---|
| Lanes 1, 3, 6 and 7 | Blank |
| Lanes 2 and 7 | pI markers |
| Lane 4 | Immuno-affinity purified product from |
| | R223 grown in glutamine-containing |
| | Iscove's medium |
| Lane 5 | Immuno-affinity purified product from |
| | clone 3E10 of GS-transfected R223 cell |
| | line grown in glutamine-free Iscove's |
| | medium |

For the product from R223 there are at least 13 visible bands, spread across the length of the gel. For the product produced by the GS transfected cell line 3E10, the more basic bands (resolved at the top of the gel) are much less intense that for the product from cell line R223, while the more acidic bands (at the bottom of the gel) are increased in intensity for cell line 3E10. There is also at least one extra acidic band detectable in the product made using the GS-transfectant 3E10 that is not detectable in product from the parent line R223. This indicates an increased degree of sialylation for the product made from the GS-transfected line 3E10.

TABLE 12
Growth and productivity of GS transfectant 3E10 cells in
serum-free suspension culture in Iscove's medium

| | Maximum viable | EPO | qₑₚₒ |
|---|---|---|---|
| | cells/mL | EU/mL | EU/10⁶ cells/h |
| 3E10 grown in glutamine- | 0.55 | 1340 | 11.5 |
| free Iscove's medium | | | |
| R223 grown in Iscove's | 0.72 | 993 | 7.3 |
| medium containing | | | |
| glutamine | | | |

TABLE 13
Reduction in ammonia production by GS transfectant 3E10 cells in
serum-free suspension culture in Iscove's medium

| | | | qₐₘₘₒₙᵢₐ |
|---|---|---|---|
| | Maximum viable | Ammonia | nMoles/ |
| | cells/mL | mM | 10⁶ cells/h |
| 3E10 grown in | 0.55 | 0.61 | 2.6 |
| glutamine-free Iscove's | | | |
| medium | | | |
| R223 grown in Iscove's | 0.72 | 2.39 | 17.4 |
| medium containing | | | |
| glutamine | | | |

The gel data from FIG. 5 was further quantified by scanning the IEF-gel photograph densiometrically. The relative proportion of product represented by each band was quantified. Data are summarised in Table 14, where the relative proportion of each band is expressed as a percentage of the total product on the respective lane of the gel. The more acidic bands (8 to 14) possess the highest biological activity, while the less acidic bands have relatively little or no activity. It is conspicuous from the gel (FIG. 5) and the densiometric scanning chromatograms (FIG. 6, for gel lane 4 and FIG. 7, for gel lane 5) that product from the GS-transfected cell line, GSR223, is enriched in the more acidic isoforms. For the non-transfected cell line, R223, bands 8 to 14 represent 44% of the total product, while for the GS-transfected cell line, GSR223, this proportion is increased to 73%. In FIG. 6 and 7, peaks are allocated identifier numbers according to the numbering of gel bands in FIG. 5.

| band | | | | | | |
|---|---|---|---|---|---|---|
| num- | R223 | | | GS transfected cell line GSR223 | | |
| ber | percent of total | | | percent of total | | |
| 1 | | 20.626 | Total | | 1.185 | Total |
| 2 | | 9.604 | for bands | | 1.346 | for bands |
| 3 | | 3.291 | 1 to 7 = | | 1.913 | 1 to 7 = |
| 4 | | 1.934 | 55.735% | | 2.54 | 26.865% |
| 5 | | 4.593 | | | 3.362 | |
| 6 | | 7.870 | | | 7.265 | |
| 7 | | 7.817 | | | 9.254 | |
| 8 | | 15.905 | Total | | 18.347 | Total |
| 9 | | 13.384 | for bands | | 18.87 | for bands |
| 10 | | 8.103 | 8 to 14 = | | 14.339 | 8 to 14 = |
| 11 | | 5.522 | 44.263% | | 11.978 | 73.137% |
| 12 | | 1.349 | | | 7.609 | |
| 13 | | 0 | | | 1.482 | |
| 14 | | 0 | | | 0.512 | |
| | Total = | 99.998% | | Total = | 100.002% | |

1. A glutamine-auxotropic human cell transfected with an exogenous DNA sequence encoding a protein or an exogenous DNA sequence capable of altering the expression of an endogenous gene encoding a protein and an exogenous DNA sequence encoding a glutamine synthetase, wherein these exogenous DNA sequences are located on one or more than one DNA construct, said transfected cell capable of producing said protein
and capable of growing in a glutamine-free medium.

2. The glutamine-auxotrophic human cell of claim 1, wherein the exogenous DNA sequences are located on more than one DNA construct.

3. The glutamine-auxotrophic human cell of any of claim 1, wherein the glutamine-auxotrophic human cell is an immortalized glutamine-auxotrophic human cell.

4. The glutamine-auxotrophic human cell of claim 3, wherein the immortalized glutamine-auxotrophic human cell is a human fibrosarcoma cell.

5. The glutamine-auxotrophic human cell of claim 4, wherein the human fibrosarcoma cell is a HT1080 cell line.

6. The glutamine-auxotrophic human cell of claim 1, wherein the transfected cell is anchorage-independent and capable of growing in suspension in serum-free, glutamine-free medium.

7. A process for the production of a protein comprising the steps of
a) culturing a glutamine-auxotrophic human cell according to claim 1 in a culture medium under conditions suitable for expression of said protein and
b) recovering said protein.

8. The process of claim 7 wherein the protein is a glycosylated protein.

9. The use of a glutamine synthetase as a selectable marker in glutamine-auxotrophic human cells.
